(19) United States
(12) Patent Application Publication
(10) Pub. No.: US 2007/0101463 A1
(43) Pub. Date: May 3, 2007

(54) Use of porous membrane to support developing conifer somatic embryos

(75) Inventors: Pramod K. Gupta, Federal Way, WA (US); Diane G. Holmstrom, Sumner, WA (US); Bonnie Larson, Granite Falls, WA (US); Susan D. Rayfield, Auburn, WA (US); Anthony P. Swanda, Federal Way, WA (US)

Correspondence Address: WEYERHAEUSER COMPANY;INTELLECTUAL PROPERTY DEPT., CH 1J27, P.O. BOX 9777, FEDERAL WAY, WA, 98063, US

(73) Assignee: Weyerhaeuser Co., Federal Way, WA (US)

(21) Appl. No.: 11/548,937

(22) Filed: Oct. 12, 2006

Related U.S. Application Data
(60) Provisional application No. 60/731,540, filed on Oct. 27, 2005.

(51) Int. Cl. C12N 5/04; A01H 7/00

(52) U.S. Cl. 800/319; 435/419

(57) ABSTRACT

The present invention provides methods for developing conifer cotyledonary somatic embryos. In some embodiments, the methods of the invention include the step of culturing conifer pre-cotyledonary somatic embryos on a porous membrane, that is at least intermittently contacted with liquid development medium, for a period of time sufficient to produce conifer, cotyledonary, somatic embryos from the pre-cotyledonary somatic embryos.

### CROSS REFERENCE TO RELATED APPLICATION

The present application claims the benefit of U.S. Provisional Application No. 60/731,565, filed Oct. 27, 2005.

### FIELD OF THE INVENTION

The present invention relates to methods for producing plant embryos in vitro, and optionally producing plants from the plant embryos.

### BACKGROUND OF THE INVENTION

The demand for coniferous trees, such as pines and firs, to make wood products continues to increase. One proposed solution to the problem of providing an adequate supply of coniferous trees is to identify individual coniferous trees that possess desirable characteristics, such as a rapid rate of growth, and to produce numerous, genetically identical, clones of the superior trees by somatic cloning.

Somatic cloning is the process of creating genetically identical trees from tree somatic tissue. Tree somatic tissue is tree tissue other than the male and female gametes. In one approach to somatic cloning, tree somatic tissue is cultured in an initiation medium which includes hormones, such as auxins and/or cytokinins, that initiate formation of embryogenic cells that are capable of developing into somatic embryos. The embryogenic cells are then further cultured in a maintenance medium that promotes multiplication of the embryogenic cells to form pre-cotyledonary embryos (i.e., embryos that do not possess cotyledons). The multiplied embryogenic cells are then cultured in a development medium that promotes development of cotyledonary somatic embryos which can, for example, be placed within artificial seeds and sown in the soil where they germinate to yield conifer seedlings. The seedlings can be transplanted to a growth site for subsequent growth and eventual harvesting to yield lumber, or wood-derived products. The cotyledonary somatic embryos can also be germinated in a germination medium, and thereafter transferred to soil for further growth.

A continuing problem with somatic cloning of conifer embryos is stimulating efficient formation of somatic embryos that are capable of germinating to yield plants. Preferably conifer somatic embryos, formed in vitro, are physically and physiologically similar, or identical, to conifer zygotic embryos formed in vivo in conifer seeds. There is, therefore, a continuing need for methods for producing viable conifer somatic embryos from conifer embryogenic cells.

### SUMMARY OF THE INVENTION

The present inventors have discovered that a porous membrane, such as a nylon membrane, can be used to support plant tissue during the development phase of plant somatic embryo production. The developing somatic embryos are disposed on a porous membrane which is either continuously or intermittently contacted with liquid development medium. For example, the porous membrane may be placed on an absorbent pad which is soaked in development medium so that the development medium passes through the nylon membrane and contacts the embryos. The nylon membrane bearing embryos is typically enclosed within a sealed space which contains a humid atmosphere that ensures that the embryos remain moist. The embryos should not be completely submerged in development medium. The present application describes a representative system for intermittently wetting the lower surface of a membrane that bears developing somatic embryos on its upper surface. Preferred porous membranes are sufficiently strong to resist tearing when the membranes are lifted in order to transfer somatic embryos from the development stage to subsequent stages of the somatic embryo production process.

Accordingly, in one aspect, the present invention provides methods for developing conifer cotyledonary somatic embryos. The methods of this aspect of the invention each include the step of culturing conifer pre-cotyledonary somatic embryos on a porous membrane, that is at least intermittently contacted with liquid development medium, for a period of time sufficient to produce conifer, cotyledonary, somatic embryos from the pre-cotyledonary somatic embryos.

In another aspect, the present invention provides methods for producing conifer cotyledonary somatic embryos, wherein the methods each include the steps of: (a) culturing conifer somatic cells in, or on, an induction medium to yield embryogenic cells; (b) culturing the embryogenic cells prepared in step (a) in, or on, a maintenance medium to form pre-cotyledonary conifer somatic embryos; and (c) culturing pre-cotyledonary conifer somatic embryos formed in step (b) on a porous membrane, that is at least intermittently contacted with liquid development medium, for a period of time sufficient to produce conifer, cotyledonary, somatic embryos from the pre-cotyledonary somatic embryos.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a representative system for intermittently or continuously wetting a porous membrane with liquid development medium, wherein the membrane supports developing plant somatic embryos.

### DETAILED DESCRIPTION OF THE PREFERRED EMBODIMENT

Unless specifically defined herein, all terms used herein have the same meaning as they would to one skilled in the art of the present invention.

Unless stated otherwise, all concentration values that are expressed as percentages are weight per volume percentages.

In one aspect the present invention provides methods for developing conifer cotyledonary somatic embryos. The methods of this aspect of the invention each include the step of culturing conifer pre-cotyledonary somatic embryos on a porous membrane, that is at least intermittently contacted with liquid development medium, for a period of time sufficient to produce conifer, cotyledonary, somatic embryos from the pre-cotyledonary somatic embryos.

The methods of the invention can be used to produce cotyledonary somatic embryos from any conifer, such as members of the genus Pinus, such as Loblolly pine (Pinus taeda) and Radiata pine. Again, by way of example, Douglas-fir cotyledonary somatic embryos can be produced by the methods of the invention.

Membranes that are useful in the practice of the present invention are porous, have no matrix potential (or substantially no matrix potential), and are sterilizable. Examples of useful membranes include nylon membranes, nylon fiber, wire mesh, plastic mesh and polymeric fibers that do not absorb development medium. Useful pore diameters in the porous membranes are in the range of from about 5 microns to about 1200 microns, such as from about 50 microns to about 500 microns.

The development medium is a liquid medium. The development medium contains nutrients that sustain the somatic embryos. Maltose may be included in the development medium as the principal or sole source of sugar for the somatic embryos. Useful maltose concentrations are within the range of from about 1% to about 2.5%. Suitable development media typically do not include multiplication hormones, such as auxins and cytokinins.

The osmolality of the development medium can be adjusted to a value that falls within a desired range, such as from about 250 mM/Kg to about 450 mM/Kg. Typically, an osmolality of 350 mM or higher is advantageous. An example of a suitable development medium is described in Example 1 herein.

By way of example, pre-cotyledonary conifer somatic embryos may be cultured on a nylon membrane, that is at least intermittently contacted with development medium, for a period of from 5 weeks to 12 weeks, such as from 8 weeks to 10 weeks, at a temperature of from 10° C. to 30° C., such as from 15° C. to 25° C., or such as from 20° C. to 23° C.

Liquid development media can, for example, be applied to an absorbent substrate, such as a substrate made from cellulose (e.g., cellulose fibers), such as one or more filter papers, or some other absorbent paper material. The substrate absorbs the liquid development medium which passes through a porous membrane disposed on the substrate and contacts conifer precotyledonary somatic embryos disposed on the nylon membrane. The development medium promotes the development of the conifer precotyledonary somatic embryos to form cotyledonary somatic embryos.

Again, by way of example, the porous membrane can be contacted with liquid development medium using an atomiser which sprays the porous membrane with development medium. Typically, the somatic embryos are disposed on an upper surface of the membrane and the opposite, lower, surface of the membrane is sprayed with liquid development medium. By way of further example, the porous membrane bearing somatic embryos can be disposed over liquid development medium that includes a rotating stir bar which rotates sufficiently fast to spray liquid development medium up onto the lower surface of the porous membrane.

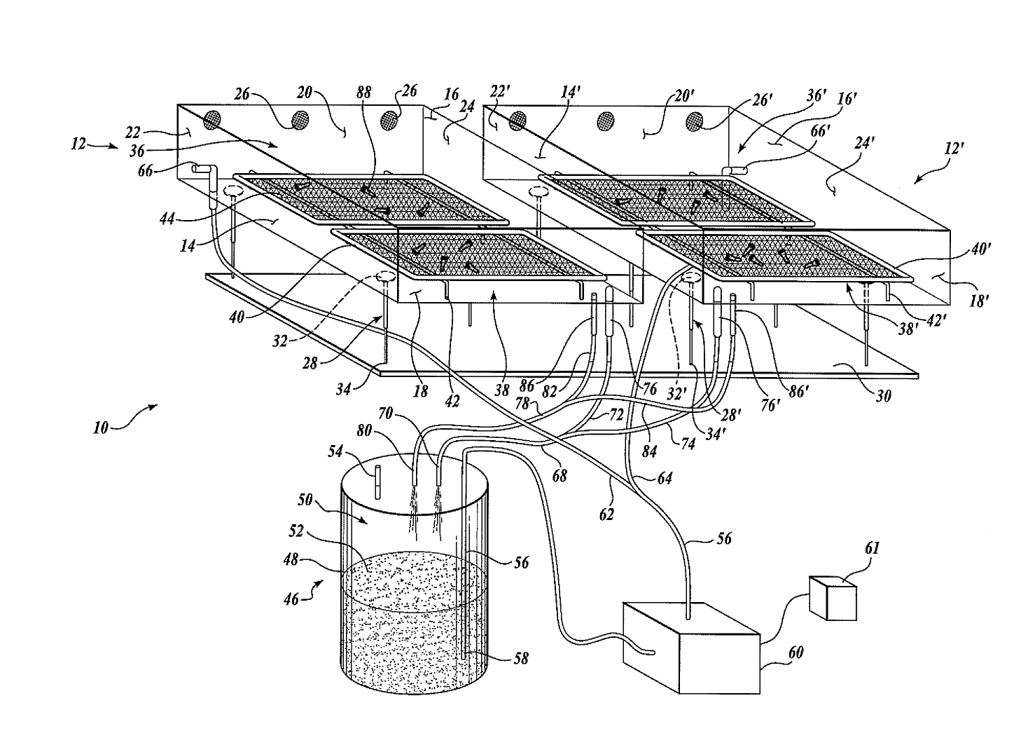

FIG. 1 shows a representative system 10 for continuously or intermittently wetting a porous membrane with development medium, wherein the membrane supports plant somatic embryos. System 10 includes a first chamber 12 including a lower surface 14, an upper surface 16, a front end 18, a rear end 20, a first side 22, and a second side 24. Rear end 20 defines three air vents 26. First chamber 12 is supported by four leg members 28 that each extend vertically from lower surface 14 of first chamber 12 to a base plate 30. Each leg member 28 includes a proximal end 32, attached to lower surface 14 of first chamber 12, and a distal end 34 that rests upon base plate 30. Distal end 34 of each leg member 28 can be rotatably adjusted about the longitudinal axis of leg member 28 to adjust the height of first chamber 12 relative to base plate 30.

Upper surface 16, lower surface 14, front end is, rear end 20, first side 22, and second side 24 together define a first chamber cavity 36. Disposed within first chamber cavity 36 are two frames 38 that each include a frame body 40 that is supported by four vertically oriented frame legs 42. Stretched across each frame 38 is a horizontally oriented, porous, nylon membrane 44.

System 10 includes a second chamber 12′ that includes the same components as first chamber 12. Components of second chamber 12′ have the same number as the corresponding component in first chamber 12, except that the component number used in connection with second chamber 12′ includes a prime (′).

Thus, second chamber 12′ includes a lower surface 14′, an upper surface 16′, a front end 18′, a rear end 20′, a first side 22′, and a second side 24′. Rear end 20′ defines three air vents 26′. Second chamber 12′ is supported by four leg members 28′ that each extend vertically from lower surface 14′ of first chamber 12′ to a base plate 30′. Each leg member 28′ includes a proximal end 32′, attached to lower surface 14′ of second chamber 12′, and a distal end 34′ that rests upon base plate 30′. Distal end 34′ of each leg member 28′ can be rotatably adjusted about the longitudinal axis of leg member 28′ to adjust the height of second chamber 12′ relative to base plate 30′.

Upper surface 16′, lower surface 14′, front end 18′, rear end 20′, first side 22′, and second side 24′ together define a second chamber cavity 36′. Disposed within second chamber cavity 36′ are two frames 38′ that each include a frame body 40′ that is supported by four vertically oriented frame legs 42′. Stretched across each frame 38′ is a horizontally oriented, porous, nylon membrane 44′.

System 10 also includes a development medium reservoir 46 that includes a reservoir body 48 that defines a cavity 50. An amount of liquid development medium 52 is disposed within cavity 50. An air vent 54 penetrates medium reservoir body 48. System 10 also includes a medium outlet tube 56 that includes a proximal end 58 that is submerged within development medium 52 within reservoir cavity 50. Outlet tube 56 is connected to a pump 60 that is controlled by a timer 61. Timer 61 is optionally programmable.

Outlet tube 56 bifurcates to form an outlet tube first portion 62 and an outlet tube second portion 64. Outlet tube first portion 62 is connected to a first development medium outlet 66 that penetrates lower surface 14 of first chamber 12 and extends into first chamber cavity 36. Outlet tube second portion 64 connects to a second development medium outlet 66′ that penetrates lower surface 14′ of second chamber 12′ and extends into second chamber cavity 36′.

System 10 also includes a first drainage tube 68 having a proximal end 70 located within medium reservoir cavity 50. First drainage tube 68 bifurcates to form a first drainage tube first portion 72 and a first drainage tube second portion 74. First drainage tube first portion 72 connects to a first medium outlet 76 that penetrates lower surface 14 of first chamber 12. First drainage tube second portion 74 connects to a first medium outlet 76′ that penetrates lower surface 14′ of second chamber 12′.

System 10 also includes a second drainage tube 78 having a proximal end 80 located within medium reservoir cavity 50. Second drainage tube 78 bifurcates to form a second drainage tube first portion 82 and a second drainage tube second portion 84. Second drainage tube first portion 82 connects to a second medium outlet 86 that penetrates (and is flush with) lower surface 14 of first chamber 12. Second drainage tube second portion 84 connects to a second medium outlet 86′ that penetrates (and is flush with) lower surface 14′ of second chamber 12′.

Developing pine somatic embryos 88 are shown disposed on nylon membrane 44.

In operation, pump 60 moves development medium 52 from reservoir 46 through medium outlet tube 56 into first chamber 12 and second chamber 12′ via outlet tube first portion 62 and outlet tube second portion 64, respectively. Pumped development medium 52 rises to the level of first medium outlet 76 and 76′, and drains therethrough into first drainage tube first portion 72 and first drainage tube second portion 74, respectively, which direct the development medium back into medium reservoir 46. Pump 60 may operate continuously or intermittently. The level of development medium 52 within first chamber 12 and second chamber 12′ is typically sufficiently high so that development medium 52 contacts nylon membrane 44, and thereby wets a portion of each somatic embryo 88 disposed on nylon membrane 44. Somatic embryos 88 should not be completely immersed in development medium 52. A humid atmosphere within reservoir cavities 50 and 50′ continuously moistens somatic embryos 88.

When pump 60 is inactivated, development medium 52 drains from first chamber 12 and second chamber 12′ through second medium outlets 86 and 86′ and is directed back into reservoir 46 via second drainage tube 78.

First medium outlets 76 and 76′ are each is set at a height within first chamber 12 and second chamber 12′, respectively, that corresponds to the desired level of development medium 52 within first chamber 12 and second chamber 12′. Typically the height of first medium outlets 76 and 76′ is the same as, or slightly greater than, the distance of nylon membranes 44 and 44′ from first chamber lower surface 14 and second chamber lower surface 14′, respectively. Consequently, while pump 60 is activated, at least a portion of each developing somatic embryo 88 is contacted with development medium 52. Pump 60 includes a progammable timer that activates and deactivates pump 60.

Second medium outlets 86 and 86′ are flush with first chamber lower surface 14 and second chamber lower surface 14′, respectively, so that when pump 60 is inoperative development medium 52 completely, or almost completely, drains from first chamber 12 and second chamber 12′.

In some embodiments, the present invention provides methods for producing conifer cotyledonary somatic embryos, wherein the methods each include the steps of: (a) culturing conifer somatic cells in, or on, an induction medium to yield embryogenic cells; (b) culturing the embryogenic cells prepared in step (a) in, or on, a maintenance medium to form pre-cotyledonary conifer somatic embryos; and (c) culturing pre-cotyledonary conifer somatic embryos formed in step (b) on a porous membrane, that is at least intermittently contacted with liquid development medium, for a period of time sufficient to produce conifer, cotyledonary, somatic embryos from the pre-cotyledonary somatic embryos. The conifer somatic cells, and resulting cotyledonary somatic embryos, can be genetically-identical.

Thus, in some embodiments, conifer somatic cells are cultured in, or on, an induction medium to yield embryogenic cells. Embryogenic cells are capable of producing one or more cotyledonary conifer somatic embryos. Examples of embryogenic cells are embryonal suspensor masses (ESMs). The induction medium typically includes inorganic salts and organic nutrient materials. The osmolality of the induction medium is typically about 160 mM/kg or even lower, but it may be as high as 170 mM/kg. The induction medium typically includes growth hormones. Examples of hormones that can be included in the induction medium are auxins (e.g., 2,4-dichlorophenoxyacetic acid (2,4-D)) and cytokinins (e.g., 6-benzylaminopurine (BAP)). Auxins can be utilized, for example, at a concentration of from 1 mg/L to 200 mg/L. Cytokinins can be utilized, for example, at a concentration of from 0.1 mg/L to 10 mg/L.

The induction medium may contain an adsorbent composition, especially when very high levels of growth hormones are used. The adsorbent composition can be any composition that is not toxic to the embryogenic cells at the concentrations utilized in the practice of the present methods, and that is capable of adsorbing growth-promoting hormones, and toxic compounds produced by the plant cells during embryo development, that are present in the medium. Non-limiting examples of useful adsorbent compositions include activated charcoal, soluble poly(vinyl pyrrolidone), insoluble poly(vinyl pyrrolidone), activated alumina, and silica gel. The adsorbent composition may be present in an amount, for example, of from about 0.1 g/L to about 5 g/L. The induction medium is typically solid, and may be solidified by inclusion of a gelling agent.

Conifer somatic cells are typically cultured in, or on, an induction medium for a period of from 3 weeks to 10 weeks, such as from 6 weeks to 8 weeks, at a temperature of from 10° C. to 30° C., such as from 15° C. to 25° C., or such as from 20° C. to 23° C.

The maintenance medium may be a solid medium, or it may be a liquid medium which can be agitated to promote growth and multiplication of the embryogenic tissue. The osmolality of the maintenance medium is typically higher than the osmolality of the induction medium, typically in the range of 120-180 mM/kg. The maintenance medium may contain nutrients that sustain the embryogenic tissue, and may include hormones, such as one or more auxins and/or cytokinins, that promote cell division and growth of the embryogenic tissue. Typically, the concentrations of hormones in the maintenance medium is lower than their concentration in the induction medium.

It is generally desirable, though not essential, to include maltose as the sole, or principal, metabolizable sugar source in the maintenance medium. Examples of useful maltose concentrations are within the range of from about 1% to about 3.0%. Conifer embryogenic cells are typically transferred to fresh maintenance medium once per week.

Useful development media are described supra. After being cultured in continuous, or periodic, contact with a development medium, the cotyledonary somatic embryos can optionally be transferred to a maturation medium, and then to a stratification medium, for a further period of culture.

The methods of the invention can be used, for example, to produce clones of individual conifer trees that possess one or more desirable characteristics, such as a rapid growth rate. Thus, in one aspect, the present invention provides methods for producing a population of genetically-identical, conifer, cotyledonary, somatic embryos. The methods of this aspect of the invention each include the step of culturing genetically-identical, conifer, precotyledonary somatic embryos on a porous membrane (e.g., porous nylon membrane) that is in continuous, or periodic, contact with a development medium, for a period of time sufficient to produce genetically-identical, conifer, cotyledonary, somatic embryos from the precotyledonary somatic embryos, wherein the development medium passes through the porous membrane and contacts the somatic embryos.

The conifer cotyledonary somatic embryos produced using the methods of the invention can optionally be germinated to form conifer plants which can be grown into coniferous trees, if desired. The cotyledonary embryos may also be disposed within artificial seeds for subsequent germination. The conifer cotyledonary somatic embryos can be germinated, for example, on a solid germination medium, such as the germination medium described in Example 1 herein. The germinated plants can be transferred to soil for further growth. For example, the Terminated plants can be planted in soil in a greenhouse and allowed to grow before being transplanted to an outdoor site. Typically, the conifer cotyledonary somatic embryos are illuminated to stimulate germination. Typically, all the steps of the methods of the invention, except germination, are conducted in the dark.

In another aspect, the present invention provides systems for developing plant somatic embryos, wherein each system includes: (a) a medium reservoir containing liquid development medium; (b) a culture chamber comprising a body defining a development medium inlet and a development medium outlet, wherein the development medium outlet is connected to the medium reservoir; (c) a porous membrane disposed on a membrane support within the culture chamber; and (d) a pump that is connected to the medium reservoir and to the development medium inlet of the culture chamber, wherein, in operation, the pump moves development medium from the reservoir to the culture chamber through the development medium inlet, and the development medium drains from the culture chamber through the development medium outlet and returns to the development medium reservoir. An example of a system of the present invention is shown in FIG. 1. The system may optionally include a timer (e.g., a programmable timer) that is connected (e.g., electrically connected) to the pump, and that activates and deactivates the pump.

In the systems of the present invention, the development medium outlet is spaced relative to the membrane support so that the development medium does not completely cover developing plant somatic embryos disposed on the porous membrane (i.e., the development medium outlet permits the development medium to drain out of the culture chamber before the medium completely submerges the embryos disposed on the porous membrane).

The following examples are provided for the purpose of illustrating, not limiting, the invention.

### EXAMPLE 1

This Example shows a representative method of the invention for producing somatic pine embryos from loblolly pine.

Female gametophytes containing zygotic embryos are removed from seeds four to five weeks after fertilization. The seed coats are removed but the embryos are not further dissected out of the surrounding gametophyte other than to excise the nucellar end. The cones were stored at 4° C. until used. Immediately before removal of the immature embryos the seeds are sterilized utilizing an initial washing and detergent treatment followed by a ten minute sterilization in 15% H₂O₂. The explants were thoroughly washed with sterile distilled water after each treatment.

Tables 1 and 2 set forth the compositions of media useful for producing pine somatic embryos.

TABLE 1
Pinus Taeda Basal Medium (BM)

| Constituent | Concentration (mg/L) |
|---|---|
| NH₄NO₃ | 150.0 |
| KNO₃ | 909.9 |
| KH₂PO₄ | 136.1 |
| Ca(NO₃)₂•4H₂O | 236.2 |
| CaCL₂•4H₂O | 50.0 |
| MgSO₄•7H₂O | 246.5 |
| Mg(NO₃)₂•6H₂O | 256.5 |
| MgCl₂•6H₂O | 50.0 |
| KI | 4.15 |
| H₃BO₃ | 15.5 |
| MnSO₄•H₂O | 10.5 |
| ZnSO₄•7H₂O | 14.4 |
| NaMoO₄•2H₂O | 0.125 |
| CuSO₄•5H₂O | 0.125 |
| CoCl₂•6H₂O | 0.125 |
| FeSO₄•7H₂O | 27.86 |
| Na₂EDTA | 37.36 |
| Maltose | 30,000. |
| myo-Inositol | 200 |
| Casamino acids | 500 |
| L-Glutamine | 1000 |
| Thiamine•HCl | 1.00 |
| Pyridoxine•HCl | 0.50 |
| Nicotinic acid | 0.50 |
| Glycine | 2.00 |
| Gelrite⁺ | 1600 |
| pH adjusted to 5.7 | |

⁺Used if a solid medium is desired.

TABLE 2
Composition of Media for Different Stage Treatments

| | |
|---|---|
| BM₁-Induction | BM + 2,4-D (15 μM) + Kinetin (2 μM) + |
| Medium | BAP (2 μM). |
| BM₂-Maintenance | BM + 2.4-D (5 μM) + Kinetin (0.5 μM) + |
| Medium | BAP (0.5 μM). GELRITE (1600 mg/L) is added |
| | when a solid medium is desired. |
| BM₃-Development | BM + 25 mg/L abscisic acid + 12% PEG-8000 + |
| Medium | 800 mg/L additional myo-inositol + 0.1% |
| | activated charcoal + 1% glucose, + 2.5% |
| | Maltose. The following amino acid mixture is |
| | added: L-proline (100 mg/L), L-asparagine (100 |
| | mg/L), L-arginine (50 mg/L), L-alanine (20 mg/L), |
| | and L-serine (20 mg/L). GELRITE (2500 mg/L) is |
| | added when a solid medium is desired. |
| BM₅- | BM₃ modified by omitting abscisic |
| Stratification | acid, and PEG-8000. GELRITE (2500 mg/L) is |
| Medium | added when a solid medium is desired. |
| BM₆-Germination | BM modified by replacing maltose with 2% sucrose. |
| Medium | Myo-inositol is reduced to 100.0 mg/L, glutamine |
| | and casamino acids are reduced to 0.0 mg/L. |
| | FeSO₄•7H₂O is reduced to 13.9 mg/L and |
| | Na₂EDTA reduced to 18.6 mg/L. Agar at 0.8% and |
| | activated charcoal at 0.25% are added. |

Induction: Sterile gametophytes with intact embryos are placed on a solid BM₁ culture medium and held in an environment at 22°-25 C with a 24 hour dark photoperiod for a time of 3-5 weeks. The length of time depends on the particular genotype being cultured. At the end of this time a white mucilaginous mass forms in association with the original explants. Microscopic examination typically reveals numerous early stage embryos associated with the mass. These are generally characterized as having a long thin-walled suspensor associated with a small head with dense cytoplasm and large nuclei.

Osmolality of the induction medium may in some instances be as high as 150 mM/kg. Normally it is about 120 mM/kg or even lower (such as 110 mM/kg).

Maintenance and Multiplication: Early stage embryos removed from the masses generated in the induction stage are first placed on a BM₂ gelled maintenance and multiplication medium. This differs from the induction medium in that the growth hormones (both auxins and cytokinins) are reduced by at least a full order of magnitude. Osmolality of this medium 130 mM/kg or higher (typically within the range of about 120-150 mM/kg for Pinus taeda) The temperature is again 22°-25 C in the dark. Embryos are cultured 12-14 days on the BM₂ solid medium before transferring to a liquid medium for further subculturing. This liquid medium has the same composition as BM₂, but lacks the gellant. The embryos at the end of the solid maintenance stage are typically similar in appearance to those from the induction stage. After 5 to 6 weekly subcultures on the liquid maintenance medium advanced early stage embryos have formed. These are characterized by smooth embryonal heads, estimated to typically have over 100 individual cells, with multiple suspensors.

Embryo Development: embryo development is conducted using the system shown in FIG. 1.

The osmotic potential of this development medium may be raised substantially over that of the maintenance medium. It has been found advantageous to have an osmolality as high as 350 mM/kg or even higher. Development is preferably carried out in complete darkness at a temperature of 22°-25° C. until cotyledonary embryos have developed. Development time is typically several weeks, such as 7 to 12 weeks.

Stratification: Cotyledonary embryos are singulated and transferred to stratification medium BM₅. This medium is similar to development medium but lacks abscisic acid, PEG-8000, and gellan gum. Embryos are cultivated on stratification medium at between about 1° C. and about 10° C. in the dark for between three to six weeks.

Conditioning over water: The mature embryos still on their nylon membrane support are lifted from the pad and placed in a closed container over H₂O at a relative humidity of 97%, for a period of about three weeks.

Germination: The dried mature embryos were rehydrated by placing them, while still on the nylon membrane support, for about 24 hours on a pad saturated with liquid Germination medium. The embryos were then placed individually on solid BM₆ medium for germination. This is a basal medium lacking growth hormones which has been modified by reducing sucrose, myo-inositol and organic nitrogen. The embryos are incubated on BM₆ medium for about 12 weeks under environmental conditions of 23°-25° C., and a 16 hour light—8 hour dark photoperiod, until the resulting plantlets have a well developed radicle and hypocotyl and green cotyledonary structure and epicotyl.

Because of the reduced carbohydrate concentration, the osmotic potential of the germination medium is further reduced below that of the development medium. It is normally below about 150 mM/kg (such as about 100 mM/kg).

### EXAMPLE 2

This Example shows that Loblolly pine somatic embryos can be developed on porous nylon membrane disposed on cellulose pads that are soaked in liquid development medium.

Embryo Treatment: Loblolly pine genotypes A, B, and C were bulked in a large flask. 0.75 mls of cells were applied onto either Whatman No. 4 filter paper, or onto nylon membrane (SeFar Co., Product No. 0-100-44 having 100 μm pore size), disposed on a double layer of absorbent pads in a Petri plate. Each pad had a diameter of 2″ and the double layer of pads was soaked with approximately 40 mls of development medium.

After development treatment, all plates were stratified for four weeks in the cold. The embryos were then singulated on dry filter paper and suspended over water in large boxes for three weeks in order to condition the embryos. The embryos were then imbibed on liquid germination medium for 24 hours, then planted into solid germination medium. The germination boxes containing the germinating embryos were moved to the light after seven days of dark treatment.

Embryo Yield After Development Treatment: Embryos were counted at the beginning of the conditioning treatment. Embryo numbers were calculated per milliliter of cells plated for each genotype. The embryo yield for embryos developed on filter paper (combining all genotypes) was 48±28. The embryo yield for embryos developed on nylon membrane (all genotypes combined) was 55±14.

The average percentage germination for embryos (all genotypes combined) cultured on filter paper was 30±5. The average percent germination for embryos (all genotypes combined) cultured on nylon membrane was 32±7.

Thus, there was not a statistically significant difference in the numbers of embryos obtained after development, or germination, using filter paper compared to nylon membrane.

### EXAMPLE 3

This Example describes the successful use of a bioreactor to develop Loblolly pine somatic embryos on a nylon membrane that is intermittently contacted with development medium.

Loblolly pine genotype A was used. 12 ml per treatment were plated in half size Cambro boxes. Each plate had 0.5 mls of cells.

The bioreactor system shown in FIG. 1 was used to perform the experiments described in this example.

Four development treatments were used: Treatment 1 had 10% C.C. (cellulose) pad with a nylon membrane (100 μm pore size) disposed on the pad; Treatment 2 had 10% C.C. pad with filter paper (Whatman #4 fp.) instead of the nylon membrane; Treatment 3 had filter paper disposed on top of the nylon membrane (no pad); Treatment 4 had only nylon membrane (no pad, no filter paper).

Development medium was pumped into the Cambro boxes until the medium touched the nylon membrane. The medium started draining 15 minutes after pumping stopped. The pump delivered medium once every 24 hours.

The experiment was stopped after 8 weeks. Every treatment produced embryos. In particular, good quality (zygotic-like) embryos developed on the membrane that was not supported by a cellulose pad.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for developing conifer, cotyledonary, somatic embryos, the method comprising the step of culturing conifer pre-cotyledonary somatic embryos on a porous membrane, that is at least intermittently contacted with liquid development medium, for a period of time sufficient to produce conifer, cotyledonary, somatic embryos from the pre-cotyledonary somatic embryos.

2. The method of claim 1 wherein the conifer pre-cotyledonary somatic embryos consist essentially of pine pre-cotyledonary somatic embryos.

3. The method of claim 2 wherein the pine pre-cotyledonary somatic embryos consist essentially of pine embryonal suspensor masses.

4. The method of claim 1 wherein the conifer pre-cotyledonary somatic embryos consist essentially of Douglas-fir pre-cotyledonary somatic embryos.

5. The method of claim 4 wherein the Douglas-fir pre-cotyledonary somatic embryos consist essentially of Douglas-fir embryonal suspensor masses.

6. The method of claim 1 wherein the porous membrane comprises pores having an average pore diameter in the range of from 5 microns to 1200 microns.

7. The method of claim 1 wherein the porous membrane consists essentially of a material selected from the group consisting of nylon membrane, nylon fiber, wire mesh, plastic mesh and polymeric fibers that do not absorb development medium.

8. The method of claim 7 wherein the porous membrane consists essentially of nylon.

9. The method of claim 1 wherein the developing conifer pre-cotyledonary somatic embryos are cultured on the porous membrane for a period of from five weeks to twelve weeks.

10. A method for developing conifer, cotyledonary, somatic embryos, the method comprising the steps of:
(a) culturing conifer somatic cells in, or on, an induction medium to yield embryogenic cells;
(b) culturing the embryogenic cells prepared in step (a) in, or on, a maintenance medium to form pre-cotyledonary conifer somatic embryos; and
(c) culturing pre-cotyledonary conifer somatic embryos formed in step (b) on a nylon membrane, that is at least intermittently contacted with liquid development medium, for a period of time sufficient to produce conifer, cotyledonary, somatic embryos from the pre-cotyledonary somatic embryos.

11. The method of claim 10 wherein the porous membrane comprises pores having an average pore diameter in the range of from 5 microns to 1200 microns.

12. The method of claim 1 wherein the porous membrane is contacted with development medium by disposing the porous membrane on a porous substrate that contains the development medium.
